(19) United States
(12) Patent Application Publication
(10) Pub. No.: US 2025/0177991 A1
(43) Pub. Date: Jun. 5, 2025

(54) POINT-OF-CARE ANALYZERS INCLUDING A SAMPLE PREPARATION TRAY

(71) Applicant: IDEXX Laboratories, Inc., Westbrook, ME (US)

(72) Inventors: Michael Fletcher, Portland, ME (US); David Lance Connolly, Eliot, ME (US); Matthew McCown Furtney, Freeport, ME (US); Peter Pajer, Gorham, ME (US)

(73) Assignee: IDEXX Laboratories, Inc., Westbrook, ME (US)

(21) Appl. No.: 18/951,816

(22) Filed: Nov. 19, 2024

Related U.S. Application Data
(60) Provisional application No. 63/604,457, filed on Nov. 30, 2023.

(51) Int. Cl. B01L 9/00

(52) U.S. Cl. CPC B01L 9/52; B01L 2200/025; B01L 2200/16; B01L 2300/0809

(57) ABSTRACT

A point-of-care analyzer is provided including an analysis unit that receives a biological sample for analysis, the analysis unit including an interrogation device; a housing at least partially surrounding the analysis unit, the housing defining a cartridge slot for receiving a cartridge comprising the biological sample; and a tray on the housing, the tray comprising an upper surface for holding the cartridge and one or more reagent holders for holding a consumable reagent.

### CROSS-REFERENCE TO RELATED APPLICATIONS

This application claims the benefit of co-pending U.S. Provisional Patent Application Ser. No. 63/604,457, filed Nov. 30, 2023 which is hereby incorporated by reference in its entirety.

### TECHNICAL FIELD

The present specification relates to biological analyzers, and more particularly, to laboratory biological analyzers with an integrated sample preparation tray.

### BACKGROUND

Medical professionals and veterinarians often utilize biological analysis devices in a laboratory setting to evaluate biological samples and diagnose abnormalities or medical conditions associated with the biological sample. These biological analysis devices may include one or more interrogation devices such as a hematology device or a microscopy device to characterize the biological sample. To properly conduct these analyses, biological samples must generally be prepared in a certain format to be compatible with the biological analysis device. For instance, this might require preparing biological samples on glass slides, reagent tubes, or cartridges prior to characterization. However, laboratory settings are often crowded spaces and lack sufficient areas to properly prepare or format biological samples. Moreover, providing a biological analysis device with a dedicated preparation area can minimize contamination with other samples and optimize workflow for a user. Accordingly, a need exists for a novel biological analysis device with an integrated sample preparation tray for preparing biological samples.

### SUMMARY

In one embodiment, the point-of-care analyzer includes an analysis unit that receives a biological sample. The analysis unit further includes an interrogation device. Further, the point-of-care analyzer includes a housing at least partially surrounding the analysis unit. The housing defines a cartridge slot for receiving a cartridge containing a biological sample. Additionally, the point-of-care analyzer includes a tray on the housing. The tray additionally comprises an upper surface, which holds the cartridge and includes one or more reagent holders that each hold a consumable reagent.

In another embodiment, a housing of a point-of-care analyzer includes a tray, the tray includes an upper surface that defines a cartridge holding section. The cartridge holding section is configured to receive and retain a cartridge that contains a biological sample. Further, the tray also includes a pair of reagent holders provided on opposite sides of the cartridge holding section, where each reagent holder receives and retains a consumable reagent.

Additional features and advantages of the technology described in this disclosure will be set forth in the detailed description which follows, and in part will be readily apparent to those skilled in the art from the description or recognized by practicing the technology as described in this disclosure, including the detailed description which follows, the claims, as well as the appended drawings.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts a perspective view of an example point-of-care analyzer with a front housing portion of a housing removed from a rear housing portion of the housing, according to one or more embodiments shown and described herein;

FIG. 2 schematically depicts a perspective view of the point-of-care analyzer of FIG. 1 with the front housing portion attached to the rear housing portion, according to one or more embodiments shown and described herein;

FIG. 3 schematically depicts a partial rear perspective view of the housing of FIG. 2, according to one or more embodiments shown and described herein;

FIG. 4 schematically depicts a perspective view of the point-of-care analyzer of FIG. 1 with the front housing portion removed from the rear housing portion and a tray removed from the front housing portion, according to one or more embodiments shown and described herein;

FIG. 5 schematically depicts a partial top perspective view of the point-of-care analyzer of FIG. 1, according to one or more embodiments shown and described herein;

FIG. 6 schematically depicts a partial cross-sectional view of the tray and the front housing portion, according to one or more embodiments shown and described herein;

FIG. 7 schematically depicts a partial top perspective view of another tray of the point-of-care analyzer of FIG. 1, according to one or more embodiments shown and described herein;

FIG. 8 schematically depicts a top perspective view top view of the tray, according to one or more embodiments shown and described herein;

FIG. 9 schematically depicts a perspective view of another example point-of-care analyzer with a ledge located at a middle position of the housing and the tray positioned on the ledge, according to one or more embodiments shown and described herein; and

FIG. 10 schematically depicts a perspective view of another example point-of-care analyzer with a ledge located at a bottom position of the housing and the tray positioned on the ledge, according to one or more embodiments shown and described herein.

### DETAILED DESCRIPTION

In modern medical and veterinary practice, biological sample testing and analysis is performed to diagnose biological maladies and prepare treatment protocols. Biological laboratories often carry out testing and analysis using interrogation devices, like optical microscopes, hematology devices, or other biological testing instruments. The emergence of accessible, benchtop digital microscopy and hematology devices has ushered in a new era of biological testing, providing researchers with the means to closely monitor and analyze biological samples with unprecedented precision in terms of resolution, specificity, dimensionality, and scale. These types of instruments have become commonplace, to such an extent that modern biological laboratories are often crowded and may lack the space for dedicated sample preparation areas.

Due to the crowded spaces in biological laboratories and the sensitive nature of biological samples, contamination is a major concern when preparing samples for testing. In many modern analysis units with optical microscopes or hematology devices, including the embodiments discussed herein, samples are first prepared in cartridges, reagent tubes, or other sample holders prior to optical analysis. These samples are then analyzed through the sample cartridge from below when conducting the analysis. Accordingly, contaminants present two problems during preparation: contaminants can become inadvertently mixed into a biological sample and compromise test results, and they can adhere to a sample cartridge during preparation and obscure optical imaging.

To enhance the reliability of the attributes measured by optical microscopy or hematology methods, it is desirable to create preparation spaces free of contaminants for preparing biological samples. Because modern laboratories are often crowded, biological analysis instruments with integrated sample preparation areas can save space and isolate biological samples. Accordingly, embodiments of the present point-of-care analyzer are directed to an analysis unit that receives a biological sample. A housing at least partially surrounds the analysis unit and defines a cartridge slot for receiving a cartridge containing the biological sample. Furthermore, a tray is on the housing for sample preparation, wherein the tray comprises an upper surface that may hold the cartridge and one or more reagent holders that may each hold a consumable reagent.

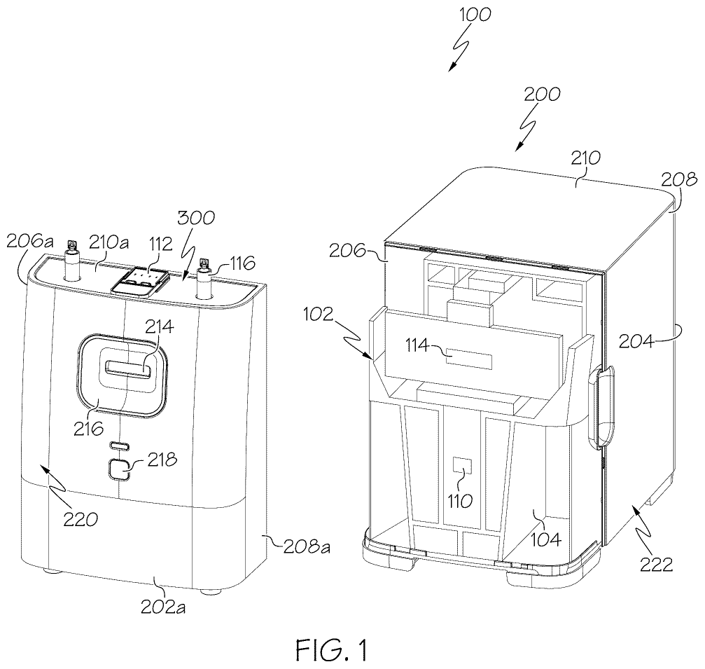

Referring now to FIG. 1, an example point-of-care analyzer 100 for analyzing a biological sample is shown consistent with a disclosed embodiment. A point-of-care analyzer 100 may include an analysis unit 102. The analysis unit 102 may include an interrogation device 104. The analysis unit 102 may further include a user input element 110 and a cartridge receptor 114 for receiving a cartridge 112. A biological sample may be contained within a chamber of the cartridge 112.

The analysis unit 102 may include one or more interrogation devices 104. The interrogation device 104 may be a microscopy device and/or a hematology device. The hematology device and the microscopy device may be configured to analyze the biological sample once received within the cartridge receptor 114. In embodiments, the hematology device and the microscopy device may analyze the biologic sample to generate attributes of the biological sample for medical diagnosis. The biological sample may be a sample of cells of a biological material. In some embodiments, the biological sample may be from a non-human animal, such as a cat, a dog, a horse, a cow, a pig, a bird, a reptile or any other suitable non-human animal. In further embodiments, the biological sample may be a blood sample, a fecal sample, a urine sample, a fine needle aspirate (FNA), lavage fluids, body cavity fluids, semen, saliva, skin swabs, skin scrapes, biopsied tissues, biological material resulting from an ear swab, or any other biological material that may be relevant to provide diagnostic medical information to a doctor, veterinarian, or lab technician.

The hematology device of the interrogation device 104 may analyze various attributes of the biologic sample. In embodiments, the hematology device may analyze a total cell count, a white blood cell count, a red blood cell count, platelet count, cell anomalies or any other suitable attributes of the biologic sample. The hematology device may have one or more parameters which are configured to be adjusted. In embodiments, an optical density, a flow rate, an extinction channel, a low angle forward light scatter channel, a right angle scatter channel, a high angle forward light scatter channel, and/or a time-of-flight channel of the hematology device may be adjusted.

The microscopy device of the interrogation device 104 may be any form of optical, electron, or scanning probe microscopy device. The microscopy device may be any one of the following: a light microscopy device, an electron microscopy device, a confocal microscopy device, a multiphoton microscopy device, or a fluorescence microscopy device. In some embodiments, the microscopy unit is a digital microscope. The microscopy unit may include, without limitations, one or more light sources, one or more lenses (such as, without limitations, a condenser lens and an objective lens), one or more mirrors and filters, one or more specimen stages, such as a well or a chamber, and any components and parts suitable for the operation of the microscopy unit. In embodiments, the analysis unit 102 may include more than one microscopy device.

The microscopy device may analyze various attributes of the biological sample. In embodiments, the microscopy device may analyze a total cell count, a white blood cell count, a red blood cell count, platelet count, cell anomalies or any other suitable attributes of the biologic sample. The microscopy device may have one or more parameters which are configured to be adjusted. In embodiments, an intensity of a light emitting device of the microscopy device may be adjusted. In further embodiments, a duration of exposure of a camera shutter of the microscopy device may be adjusted. In yet further embodiments, a magnification of a magnification device of the microscopy device may be adjusted.

The analysis unit 102 may further include a processor and a memory to communicate with the interrogation device 104, dictate operational programs to the interrogation device 104, receive measured attributes from the interrogation device 104, and communicate the measured attributes to a user. The processor may be communicatively coupled to the memory. The memory may include a non-transitory, processor-readable storage medium for storing program modules that, when executed by the processor, perform one or more processes described herein. The processor may also be communicatively coupled to the interrogation device 104 to execute processes and receive information, including measured attributes of the biological sample from the interrogation device 104. The memory may also store information communicated from the interrogation device 104, such as the hematology device or the microscopy device. The memory may be one or more memory devices that store data as well as software and may also comprise, for example, one or more of RAM, ROM, magnetic storage, or optical storage. Since disclosed embodiments may be implemented using an HTTPS (hypertext transfer protocol secure) environment, data transfer over a network, such as the Internet, may be done in a secure fashion.

The analysis unit 102 may also include a user input element 110. In some embodiments, the analysis unit 102 may include two or more user input elements 110. The user input element 110, in some embodiments, may be a power switch or power button operatively coupled to the analysis unit 102 to dictate a power state of the point-of-care analyzer 100. In other embodiments, the analysis unit 102 may further include a control device operatively coupled to the analysis unit 102. In some embodiments, the control device may include an integrated user interface display. The control device may also be a distinct device, e.g. a user's computer, tablet or phone that can communicate commands to the analysis unit 102 through a physical connection or through a remote connections like Bluetooth or data transfer via an HTTPS environment, such as the Internet.

Although a specific number of interrogation devices 104 and user input elements 110 are depicted in FIG. 1, any number of these devices may be provided. Furthermore, the functions provided by one or more devices of the analysis unit 102 may be combined and the functionality of any one or more components of the analysis unit 102 may be implemented by any appropriate computing environment.

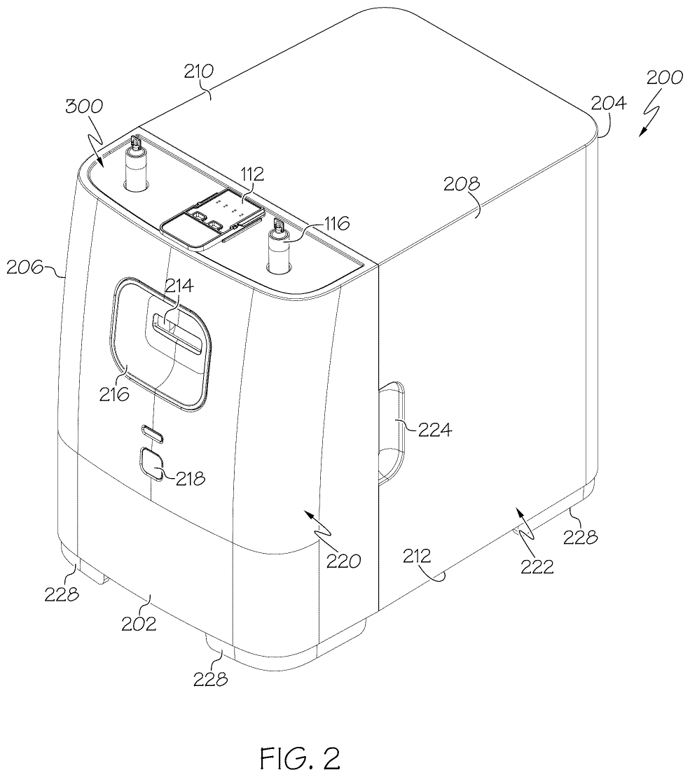

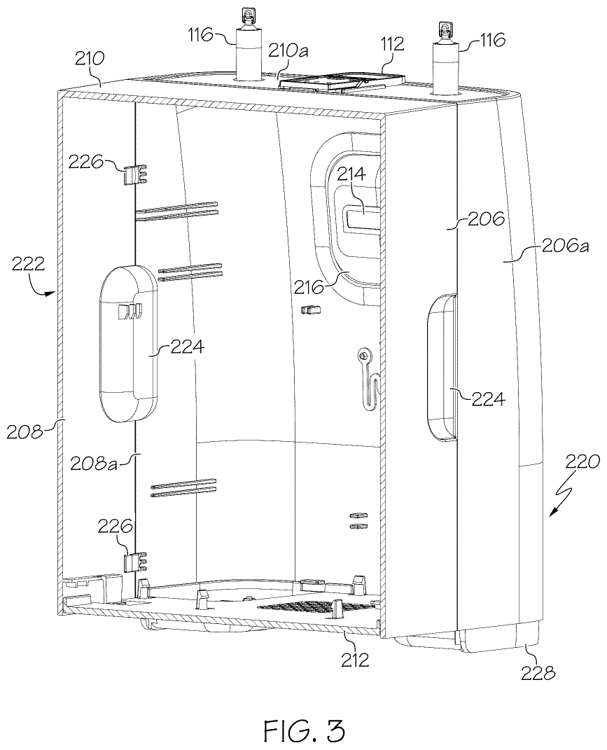

As shown in FIGS. 1 and 2, the analysis unit 102 of the point-of-care analyzer 100 is surrounded, or at least partially surrounded, by a housing 200. Referring specifically to FIG. 2, the housing 200 may include a front section 202, a rear section 204, a left side section 206, a right side section 208, an upper section 210, and a lower section 212. In embodiments, the housing 200 may also include a front housing portion 220 separable from and/or movable relative to a rear housing portion 222. The housing 200 may further include one or more access handles 224, one or more connection points 226 (FIG. 3), and one or more feet 228.

Referring again to FIG. 2, the housing 200 may be any solid or semi-solid barrier of suitable size and shape that can surround or partially surround the analysis unit 102. In embodiments, the housing 200 may completely surround the analysis unit 102. The housing 200 may be made from any suitable material including, for example, metal alloys, high performance plastics, ceramics, or composite materials comprising two or more materials. The housing 200 may comprise one or more exterior sections that define the exterior structure of the housing 200. As discussed herein, the housing 200 may include six exterior sections to form an overall cubic or a rectangular prismatic shape. The six exterior sections may include the front section 202, the rear section 204, the left side section 206, the right side section 208, the upper section 210, and the lower section 212. In embodiments, the exterior sections may meet at ninety-degree angles to form defined edges separating the exterior sections. In other embodiments, the exterior sections may meet at rounded edges. The housing 200 may include both defined and rounded edges. The housing 200 may also possess flat or substantially flat exterior sections. Alternatively, the housing 200 may include exterior sections with a rounded shape. In embodiments, the housing 200 may comprise one or more flat or substantially flat exterior sections and one or more rounded exterior sections to form the housing 200.

Referring to FIGS. 1 and 2, the housing 200 may define one or more slots, through which a user can access, communicate with, or pass biological samples to the analysis unit 102. Specifically, the housing 200 may define a cartridge slot 214. The cartridge slot 214 may be configured to have corresponding dimensions to the cartridge 112. In embodiments, the cartridge slot 214 is a rectangular opening. The cartridge slot 214 may be located at a corresponding position to align with the cartridge receptor 114. The cartridge slot 214 and cartridge receptor 114 may be arranged so that the cartridge 112 may pass through the cartridge slot 214 defined in the housing 200, and be accepted into the cartridge receptor 114 so that the interrogation device 104 can then analyze the biological sample within the cartridge 112 and communicate the measured attributes of the biological sample to the user. After analysis, or in response to user operation, the cartridge receptor 114 may eject the cartridge 112 back through the cartridge slot 214, where a user can remove the cartridge 112 from the point-of-care analyzer 100.

The housing 200 may further define a cartridge slot recess 216 encircling the cartridge slot 214. The cartridge slot recess 216 may be a concave region of the housing 200 defined on the same exterior section of the housing 200 as the cartridge slot 214. In embodiments, the cartridge slot 214 and cartridge slot recess 216 are both defined on the front section 202 of the housing 200 so that the cartridge slot 214 is disposed within a center of the cartridge slot recess 216. The cartridge slot recess 216 may extend inward from the exterior section of the housing 200 towards the analysis unit 102. The concave shape of the cartridge slot recess 216 may permit the cartridge slot 214 to be arranged in close proximity to the analysis unit 102, specifically to the cartridge receptor 114. The arrangement of the cartridge slot 214, the cartridge slot recess 216, and the cartridge receptor 114 in close proximity to one another may eliminate or substantially reduce empty space between the cartridge slot 214 and the cartridge receptor 114 to allow a user to efficiently guide the cartridge 112 into the analysis unit 102, minimizing the potential for misalignment with the cartridge receptor 114. In embodiments, the cartridge slot recess 216 and the cartridge slot 214 may be formed from a different material than the rest of the housing 200 in order to prevent scratching and scuffing on the cartridge 112 and to preserve the aesthetic appearance of the point-of-care analyzer 100.

Still referring to FIGS. 1 and 2, the housing 200 may define a user input element slot 218. The user input element slot 218 may have corresponding dimensions to the user input element 110 positioned on the analysis unit 102. The user input element slot 218 may be located at a corresponding location to align with the user input element 110. The user input element slot 218 and user input element 110 may be arranged so that a user may directly engage the user input element 110 through the housing 200. In embodiments, the user input element slot 218 may be open to permit a user to directly access the user input element 110 through the housing 200. In other embodiments, the user input element slot 218 may further comprise a switch, cover, or other intermediary mechanism that allows a user to engage the user input element 110 indirectly through the housing 200. In an embodiment, the user input element slot 218 may include a power button cover and the user input element 110 may be a power button, wherein a user can engage the power button located on the analysis unit 102 through the housing 200 by pressing the power button cover.

In embodiments, the housing 200 may further include a front housing portion 220 and a rear housing portion 222, wherein the front housing portion 220 is moveable relative to the rear housing portion 222. The front housing portion 220 and the rear housing portion 222 should not be understood to be confined to any one of the exterior sections of the housing 200 discussed herein. Both the front housing portion 220 and the rear housing portion 222 may include one or more of the exterior sections. In embodiments, the front housing portion 220 and the rear housing portion 222 may also include parts of one or more of the exterior sections.

Still referring to FIGS. 1 and 2, the front housing portion 220 may include the entirety of the front section 202, part of the left side section 206, part of the right side section 208, and part of the upper section 210. The front housing portion 220 may be movable relative to the rear housing portion 222, such that front housing portion 220 and rear housing portion 222 can be separated to expose the analysis unit 102. In embodiments, the front housing portion 220 includes a front section 202a of the front housing portion 220, a left side section 206a of the front housing portion 220, a right-side section 208a of the front housing portion 220, and an upper section 210a of the front housing portion 220. The cartridge slot 214, the cartridge slot recess 216, and the user input element slot 218 may be defined within the front section 202a of the front housing portion 220. The cartridge slot 214 may be encircled by the cartridge slot recess 216, wherein the cartridge slot 214 and the cartridge slot recess 216 are positioned above the user input element slot 218 in a vertical direction.

Referring specifically to FIG. 2, the rear housing portion 222 may include the entirety of the rear section 204 and the entirety of the lower section 212, as well as, part of the left side section 206, part of the right side section 208, and part of the upper section 210. The rear housing portion 222 may include the plurality of feet 228, discussed herein, extending downward from the lower section 212 in a vertical direction. In embodiments, the feet 228 may be made from, for example, a soft polymer, rubber, or any other suitable material that can successfully dampen vibrations that could interfere with the operations of the analysis unit 102 (FIG. 1). In further embodiments, one or more of the feet 228 may be adjustable to permit leveling of the point-of-care analyzer 100 (FIG. 1). Additionally, the rear housing portion 222 may define the one or more access handles 224 discussed herein. The access handle 224 is a recess formed in the housing 200, positioned at a junction of the front housing portion 220 and the rear housing portion 222. The access handle 224 may be any shape and size to permit a user to grasp around or under an edge of the front housing portion 220 and move the front housing portion 220 with respect to the rear housing portion 222. In embodiments, the rear housing portion 222 defines two access handles 224 positioned on opposite sides of the housing 200, with one access handle 224 defined on the left side section 206 and the other defined on the right side section 208.

Referring now to FIG. 3, the front housing portion 220 and the rear housing portion 222 may be permanently affixed at one or more connection points 226. In embodiments, the one or more connection points 226 may include hinges, flaps, pivots, or other similar mechanisms that maintain permanent contact between the front housing portion 220 and the rear housing portion 222. In other embodiments, the front housing portion 220 may be removably coupled to the rear housing portion 222, such that, when removed, the connection points 226 of the front housing portion 220 and the connection points 226 of the rear housing portion 222 are not in contact. In embodiments, the connection points 226 may include hooks, latches, magnets, or other similar mechanisms that permit removable coupling of the front housing portion 220 and the rear housing portion 222.

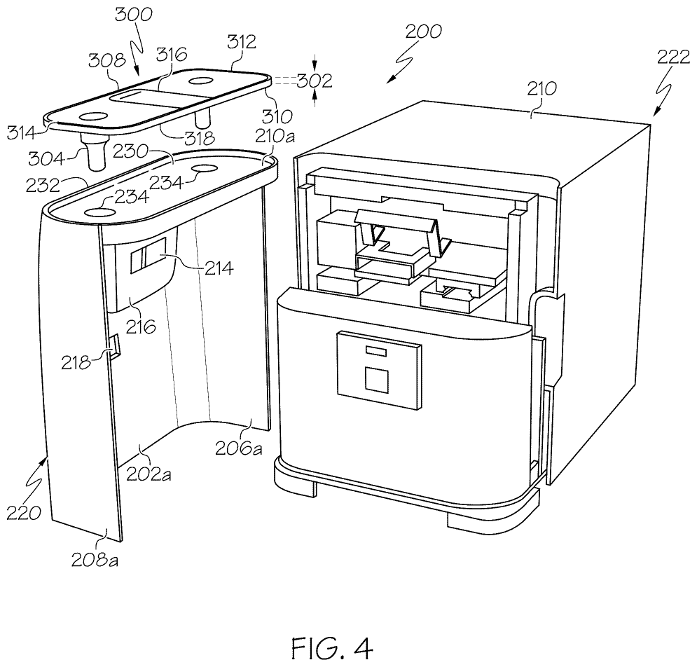

Referring now to FIG. 4, the point-of-care analyzer 100 (FIG. 1) may also include a tray 300 for sample preparation. The tray 300 may be on the housing 200 to save space in a laboratory environment. In embodiments where the housing 200 includes the front housing portion 220 and the rear housing portion 222, the tray 300 may be on the upper section 210a of the front housing portion 220 above the cartridge slot 214 in a vertical direction. In some embodiments, the tray 300 may be integrated with the housing 200 such that it is formed as a one-piece, monolithic structure with the housing 200. In other embodiments, the tray 300 may be removable with respect to the housing 200, as shown in FIG. 4, to facilitate cleaning of the tray 300 and to facilitate sample preparation in different locations within a laboratory environment.

The upper section 210 of the housing 200 may further define a recessed region 230 that may correspond to the dimensions of the tray 300 and receive the tray 300. The recessed region 230 may be formed in the upper section 210 of the housing 200 and defines a housing ridge 232 extending along the perimeter of the upper section 210 of the housing 200. In embodiments, the recessed region 230 may have a depth corresponding to a thickness 302 of the tray 300. The tray 300 may be seated in the recessed region 230 so that the height of the edges 308, 310, 312, 314 of the tray 300 corresponds to the height of the housing ridge 232. Accordingly, the tray 300, when seated within the recessed region 230, does not extend above the housing ridge 232. The recessed region 230 may further define one or more cavities 234. The cavities 234 may be positioned to accommodate protrusions 304 extending downward, in a vertical direction, from the lower surface 318 of the tray 300 and may allow the tray 300 to be seated within the recessed region 230. In embodiments, the upper section 210a of the front housing portion 220 defines the recessed region 230.

Still referring now to FIG. 4, the tray 300 may include edges 308, 310, 312, 314. The orientation and the relative positions of the edges 308, 310, 312, 314 may be defined with respect to the orientation of the point-of-care analyzer 100 (FIG. 1). In embodiments, the tray 300 may include a front edge 308, a rear edge 310 opposite the front edge 308, a first side edge 312 extending between the front edge 308 and the rear edge 310, and a second side edge 314 opposite the first side edge 312 extending between the front edge 308 and the rear edge 310. Additionally, the tray 300 may define an upper surface 316 and a lower surface 318 opposite the upper surface 316. When the tray 300 is on the housing 200, the upper surface 316 may face upward in the vertical direction such that it is exposed to the user. The lower surface 318 may face downward in the vertical direction such that it is adjacent to the upper section 210 of the housing 200 and the lower surface 318 is not exposed to the user.

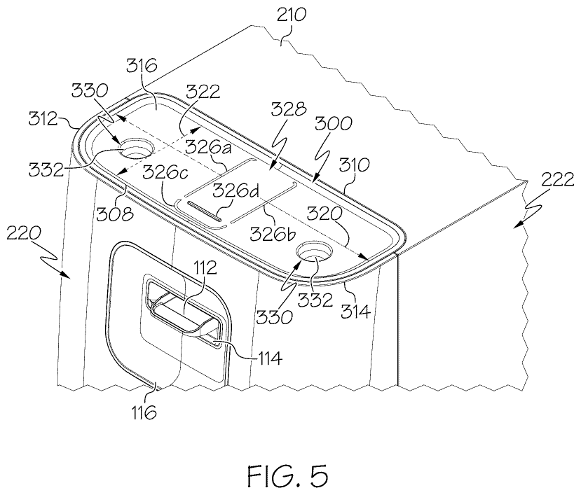

Referring to FIG. 5, the tray 300 includes the upper surface 316 of the tray 300, a plurality of ribs 326a-d, collectively referred to as ribs 326, a cartridge holding section 328, and one or more reagent holders 330. The tray 300 may include a longitudinal axis 320 that extends between the first side edge 312 and the second side edge 314. The tray 300 may further include a transverse axis 322 that extends between the front edge 308 and the rear edge 310. In embodiments, a length of the longitudinal axis 320 is longer than a length of the transverse axis 322. The upper surface 316 of the tray 300 is bounded by the edges 308, 310, 312, 314 of the tray 300, wherein the upper surface 316 of the tray 300 extends from the first side edge 312 to the second side edge 314 along the longitudinal axis 320 and extends from the front edge 308 to the rear edge 310 along the transverse axis 322. In embodiments, the upper surface 316 of the tray 300 may be positioned at a height lower than the height of the edges 308, 310, 312, 314 of the tray 300, wherein the upper surface 316 of the tray 300 is lower than the upper section 210 of the housing 200 in the vertical direction when the tray 300 is positioned on the housing 200. The lower relative positioning of the upper surface 316 of the tray 300 with respect to the edges 308, 310, 312, 314 of the tray 300 and the upper section 210 of the housing 200 creates a semi-confined region that is well-suited to contain biological material when preparing the biological material for analysis.

The tray 300 and the upper surface 316, in particular, are configured to accommodate the preparation of biological materials in the cartridge 112. The cartridge 112 may be any suitable size or shape to cooperate with the analysis unit 102 (FIG. 1) and fit within the upper surface 316 of the tray 300. The cartridge 112 may include a chamber and at least a bottom surface. In embodiments, the biological samples are analyzed by the interrogation device 104 (FIG. 1) through an optically transparent bottom surface to determine the attributes of the biological sample. Accordingly, biological samples must be carefully prepared within the chamber for accurate analysis. The biological samples may include either biological cells in a dry form or biological cells suspended in a liquid solution.

Referring again to FIG. 5, the tray 300, as discussed herein, also includes the plurality of ribs 326 arranged on the upper surface 316 of the tray 300 to accommodate a cartridge 112. The plurality of ribs 326 may extend upward from the upper surface 316 of the tray 300 and correspond to the dimensions of the cartridge 112. In embodiments, the plurality of ribs 326 includes a first rib 326a extending parallel to the transverse axis 322; a second rib 326b spaced apart from the first rib 326a and also extending parallel to the transverse axis 322; a third rib 326c located proximate to the front edge 308, between the first rib 326a and the second rib 326b, and extending parallel to the longitudinal axis 320; and a fourth rib 326d spaced apart from the third rib 326c, positioned in the center of the first rib 326a, the second rib 326b, and the third rib 326c, and extending parallel to the longitudinal axis 320. The plurality of ribs 326 may be positioned in a center of the upper surface 316 of the tray 300. The plurality of ribs 326 further define the cartridge holding section 328, wherein the cartridge 112 may be positioned and retained within the cartridge holding section 328 by the plurality of ribs 326. In embodiments, the cartridge holding section 328 is positioned in the center of the upper surface 316 of the tray 300, such that the center of the cartridge holding section 328 is equidistant from the first side edge 312 and the second side edge 314 along the longitudinal axis 320, and equidistant from the front edge 308 and the rear edge 310 along the transverse axis 322. The plurality of ribs 326 may include a pair of ribs 326 arranged parallel to and spaced apart from one another. The spacing of the pair of ribs 326 may be configured to correspond to the dimensions of the cartridge 112. In embodiments, the pair of ribs 326 may extend parallel to the front edge 308 and the rear edge 310. In other embodiments, the pair of ribs 326 may extend perpendicular to the front edge 308 and the rear edge 310. In further embodiments, the tray 300 may comprise a first pair of ribs 326 extending parallel to the front edge 308 and the rear edge 310 and a second pair of ribs 326 extending perpendicular to the front edge 308 and the rear edge 310.

The upper surface 316 of the tray 300 may also be configured to accommodate the preparation of biological materials in consumable reagents 116 (FIG. 1). Consumable reagents 116 (FIG. 1) may include tubes, vials, or other containers suited to contain a liquid solution. Liquid solutions described herein may include a biological sample combined with a liquid diluent. In embodiments, the analysis unit 102 (FIG. 1) may be configured to accept a consumable reagent 116 (FIG. 1) and analyze the biological sample therein. In other embodiments, the consumable reagents 116 (FIG. 1) may be used as preparatory tools to contain a biological sample prior to its introduction into the cartridge 112 (FIG. 1). Therefore, maintaining the consumable reagents 116 (FIG. 1) in close proximity to the cartridge 112 (FIG. 1) on the upper surface 316 of the tray 300 may be particularly desirable.

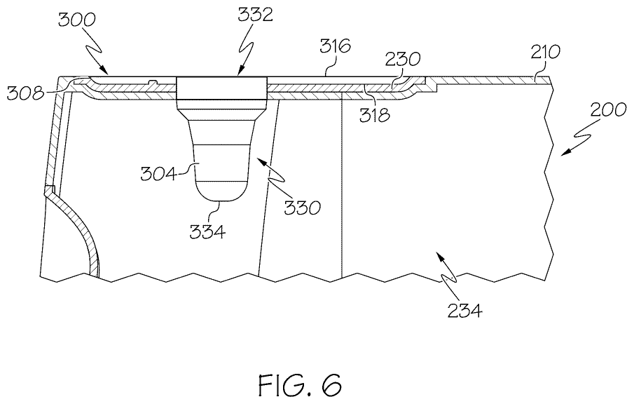

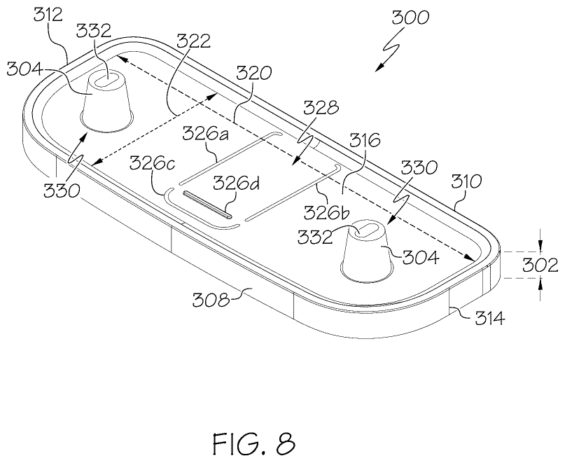

Referring now to FIGS. 6 and 8, the tray 300 may include one or more reagent holders 330 configured to receive and retain a respective consumable reagent 116 (FIG. 1). The reagent holder 330 may include a protrusion 304 extending from the upper surface 316 or the lower surface 318 of the tray 300. In embodiments, the protrusion 304 may extend upward from the upper surface 316 of the tray 300, as shown in FIG. 8, while in other embodiments the protrusion 304 may extend downward from the lower surface 318 of the tray 300, as shown in FIG. 6. The reagent holder 330 may also include a protrusion 304 extending from both the upper surface 316 of the tray 300 and the lower surface 318 of the tray 300. The reagent holder 330 may have different shapes. In embodiments, the reagent holders 330 may have a conical, hemispherical, columnar, or any other shape that is suited to receive and retain a consumable reagent 116 (FIG. 1). The regent holder 330 may further include an opening 332 and a bottom 334. The opening 332 may be spherical, ovular, rectangular, or any other shape that would permit a consumable reagent 116 (FIG. 1) to enter into the reagent holder 330 and be positionable in the opening 332. The bottom 334 of the reagent holder 330 may be flat or it may be rounded. In embodiments, as shown in FIG. 5, the one or more reagent holders 330 include a pair of reagent holders 330. The pair of reagent holders 330 may be disposed on the upper surface 316 of the tray 300 and may be spaced apart from one another and positioned on opposite sides of the upper surface 316 of the tray 300. In some embodiments, the pair of reagent holders 330 may be positioned on opposite sides of the upper surface 316 of the tray 300 such that the reagent holders 330 are positioned along the longitudinal axis 320 of the tray 300 with a first reagent holder 330 nearest to the first side edge 312 of the tray 300, a second reagent holder 330 nearest to the second side edge 314 of the tray 300, and the center of the cartridge holding section 328 arranged along the longitudinal axis 320 between the first reagent holder 330 and the second reagent holder 330. Maintaining the reagent holders 330 on opposite sides of the upper surface 316 of the tray 300 may present a benefit to users by allowing the segregation of biological samples to minimize confusion regarding the source of a biological same and reduce the chance of inaccurate results. For example, this can be useful where a user does not want to confuse samples from a left and right ear.

Referring now to FIG. 6, as described herein, the reagent holder 330 may include the protrusion 304 extending from the lower surface 318 of the tray 300. The reagent holder 330 may include the opening 332 formed at an upper end of the protrusion 304. The protrusion 304 may extend downward, in the vertical direction, into the cavity 234 of the housing 200. In embodiments, the opening 332 at the upper end of the protrusion 304 may be defined within the tray 300 so that it is maintained at the same height as the upper surface 316 of the tray 300. In embodiments, as shown in FIG. 4, the tray 300 may include a pair of reagent holders 330 wherein each reagent holder 330 includes a protrusion 304 extending from the lower surface 318 of the tray 300 and each reagent holder 330 may have an opening 332 formed at an upper end of its respective protrusion 304. FIG. 6 also illustrates the tray 300 seated within the recessed region 230 of the housing 200. As described herein, edges 308, 310, 312, 314 of the tray 300 may be flush with the upper section 210 of the housing 200. The upper surface 316 of the tray 300 may be lower than the edges 308, 310, 312, 314, such that biological sample may be prepared and contained within the upper surface 316 of the tray 300.

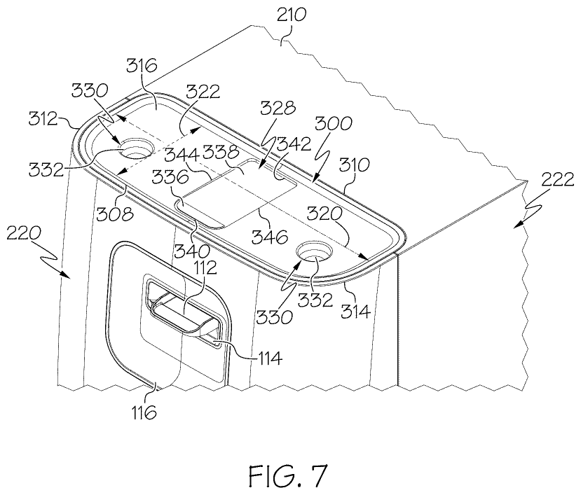

Referring now to FIG. 7, in some embodiments, the tray 300 defines the cartridge holding section 328 such that the cartridge holding section 328 is recessed within the upper surface 316 of the tray 300. The cartridge holding section 328 may be maintained at a vertical height that is lower than the upper surface 316 of the tray 300. In embodiments, the tray 300 defines one or more walls that form a perimeter of the cartridge holding section 328. In embodiments, the tray 300 defines a front wall 340, a rear wall 342, a first side wall 344, and a second side wall 346. The front wall 340 is located proximate to the front edge 308 of the tray 300 and extends parallel to the longitudinal axis 320. The rear wall 342 is located proximate to the rear edge 310 of the tray 300, spaced apart from the front wall 340, and also extends parallel to the longitudinal axis 320. The first side wall 344 is positioned on a first side of the cartridge holding section 328, extends between the front wall 340 and the rear wall 342, and extends parallel to the transverse axis 322. The second side wall 346 is spaced apart from the first side wall 344, positioned on an opposite side of the cartridge holding section 328, and extends parallel to the transverse axis 322. The walls 340, 342, 344, 346 may define the cartridge holding section 328 in a substantially central location of the upper surface 316 of the tray 300.

Additionally, the walls 340, 342, 344, 346 may be spaced apart from one another at predetermined distances to define the cartridge holding section 328 such that the cartridge 112 may be positioned and retained within the cartridge holding section 328 by the walls 340, 342, 344, 346. In embodiments, the cartridge holding section 328 may have a uniform depth along a bottom 338 of the cartridge holding section 328 extending from a base of the front wall 340 to a base of the rear wall 342. In other embodiments, the cartridge holding section 328 may further include a projection 336 extending from the bottom 338 of the cartridge holding section 328 and positioned proximate to the front wall 340, wherein the front wall 340 extends from the upper surface 316 of the tray 300 to the projection 336. The projection 336 may be maintained at a vertical height below the upper surface 316 of the tray 300, but above the bottom 338 of the cartridge holding section 328. In embodiments, the front wall 340 extends to a depth below the upper surface 316 of the tray 300 that is greater than or equal to 0.25 mm and less than or equal to 2 mm. In embodiments, the front wall 340 has a depth below the upper surface 316 of the tray 300 greater than or equal to 0.5 mm and less than or equal to 1.75 mm. In embodiments, the front wall 340 has a depth below the upper surface 316 of the tray 300 greater than or equal to 0.75 mm and less than or equal to 1.5 mm. In embodiments, the front wall 340 has a depth below the upper surface 316 of the tray 300 greater than or equal to 1 mm and less than or equal to 1.25 mm. In embodiments, the rear wall 342 has a depth below the upper surface 316 of the tray 300 greater than or equal to 1.25 mm and less than or equal to 3 mm. In embodiments, the rear wall 342 has a depth below the upper surface 316 of the tray 300 greater than or equal to 1.5 mm and less than or equal to 2.75 mm, greater than or equal to 1.75 mm and less than or equal to 2.5 mm. In embodiments, the rear wall 342 has a depth below the upper surface 316 of the tray 300 greater than or equal to 2 mm and less than or equal to 2.25 mm. Furthermore, the depth of the cartridge holding section 328 may be sloped from the projection 336 to the bottom 338 of the cartridge holding section 328.

Referring now to FIG. 8, another embodiment of the reagent holder 330 may include the protrusion 304 extending from the upper surface 316 of the tray 300. The reagent holder 330 may include an opening 332 formed at an upper end of the protrusion 304. In embodiments, as shown in FIG. 8, the tray 300 may include a pair of reagent holders 330 wherein each reagent holder 330 includes a protrusion 304 extending from the upper surface 316 of the tray 300 and each reagent holder 330 includes an opening 332 formed at an upper end of each respective protrusion 304. In embodiments, a consumable reagent 116 (FIG. 1) may be positionable within the opening 332 of each respective reagent holders 330 so that a lower portion of each consumable reagent 116 (FIG. 1) is retained within its reagent holder 330 and an upper portion of each consumable reagent 116 (FIG. 1) extends beyond the opening 332 in the vertical direction to be accessible to the user. FIG. 8 also depicts an exemplary embodiment of the tray 300 being removable from the housing 200 (FIG. 1). The tray 300 may have a uniform thickness 302 along the longitudinal axis 320 and the transverse axis 322. Alternatively, in other embodiments, the thickness 302 of the tray 300 may be varied along either the longitudinal axis 320 or the transverse axis 322. FIG. 8 also depicts an embodiment of the tray 300 wherein the upper surface 316 of the tray 300 is positioned at a height lower than the height of the edges 308, 310, 312, 314 of the tray 300 to create a semi-confined region to prepare biological materials for analysis.

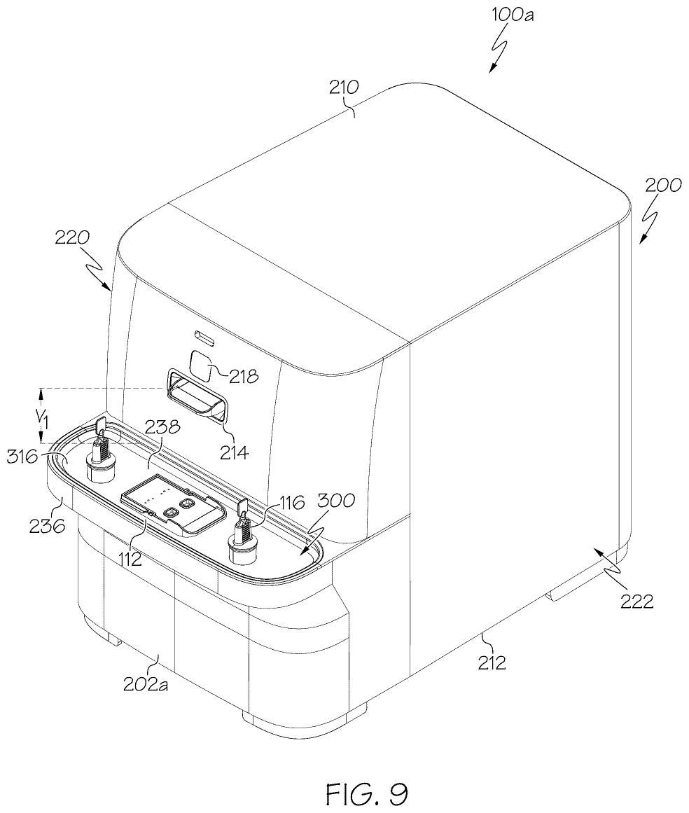

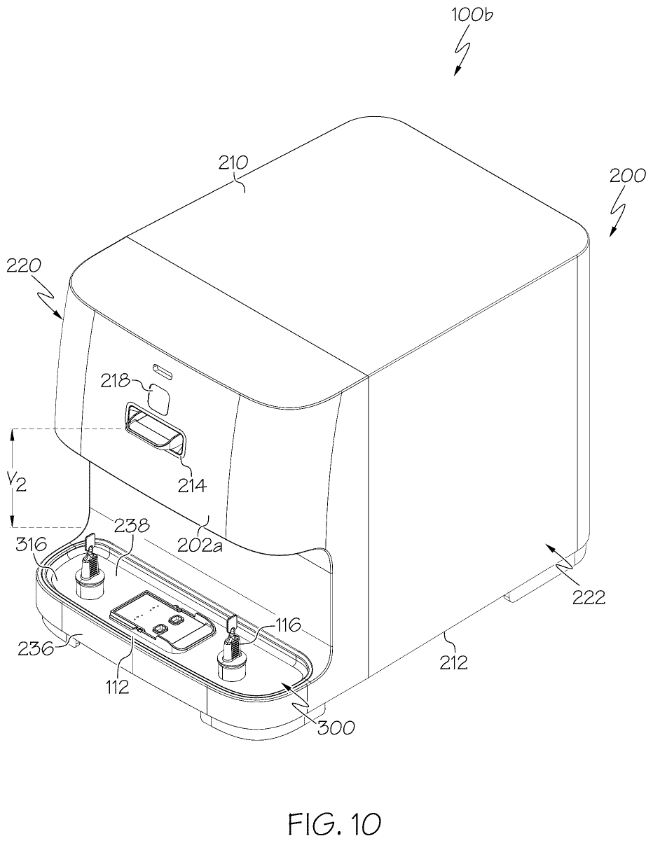

Referring now to FIGS. 9 and 10, alternative embodiments of a point-of-care analyzer 100a, 100b are depicted. However, like reference numbers will be used to refer to like parts. Particularly, the housing 200 may include a ledge 236. The ledge 236 may further include an upper face 238, wherein the tray 300 may be positioned on the upper face 238 of the ledge 236. The ledge 236 may extend in a horizontal direction from the front section 202 of the housing 200. The ledge 236 may be positioned so that the ledge 236 has a vertical position below the upper section 210 of the housing 200 and above the lower section 212 of the housing 200. In embodiments, the ledge 236 may extend in a horizontal direction from the front section 202a of the front housing portion 220. The ledge 236 may further define the recessed region 230 (FIG. 4) with a depth corresponding to the thickness 302 (FIG. 4) of the tray 300, as discussed herein. The tray 300 may be seated in the recessed region 230 (FIG. 4) so that the thickness 302 (FIG. 4) of the tray 300 does not extend above the upper face 238 of the ledge 236.

Referring now to FIG. 9, the ledge 236 may be positioned at a middle location, according to alternative embodiments of the point-of-care analyzer 100a, wherein the ledge 236 extends from the front section 202a of the front housing portion 220 in a horizontal direction. More particularly, the ledge 236 is positioned below the cartridge slot 214 in a vertical direction. Additionally, the ledge 236 may be positioned below the user input element slot 218, so that the ledge 236 is fixed below both the cartridge slot 214 and below the user input element slot 218 along a vertical axis. In embodiments, the ledge 236 may be selectively coupled to the housing 200 such that ledge 236 may be movable relative to the housing 200. Accordingly, the ledge 236 may be positionable by a user at various vertical heights on the front section 202a of the front housing portion 220, depending on the user's needs. The ledge 236 may further include the tray 300, positioned on the upper face 238 of the ledge 236. In embodiments, the tray 300 and the cartridge slot 214 may have a vertical spacing V1, wherein a distance between the upper surface 316 of the tray 300 and the cartridge slot 214 is greater than or equal to 20 mm and less than or equal to 80 mm, greater than or equal to 25 mm and less than or equal to 75 mm, greater than or equal to 30 mm and less than or equal to 70 mm, greater than or equal to 35 mm and less than or equal to 65 mm, or even greater than or equal to 40 mm and less than or equal to 60 mm.

Referring now to FIG. 10, in embodiments, the ledge 236 may be positioned at a lower location, according to alternative embodiments of the point-of-care analyzer 100b, wherein the ledge 236 extends from the front section 202a of the front housing portion 220 in a horizontal direction. More particularly, the ledge 236 is positioned below the cartridge slot 214 in a vertical direction. Additionally, the ledge 236 may be positioned below the user input element slot 218, so that the ledge 236 is fixed below both the cartridge slot 214 and below the user input element slot 218 along a vertical axis. In embodiments, the ledge 236 may be selectively coupled to the housing 200 such that ledge 236 may be movable relative to the housing 200. Accordingly, the ledge 236 may be positionable by a user at various vertical heights on the front section 202a of the front housing portion 220, depending on the user's needs. The ledge 236 may further include the tray 300, positioned on the upper face 238 of the ledge 236. In embodiments, the tray 300 and the cartridge slot 214 may have a vertical spacing V2, wherein the distance between the upper surface 316 of the tray 300 and the cartridge slot 214 is greater than or equal to 140 mm and less than or equal to 200 mm, greater than or equal to 145 mm and less than or equal to 195 mm, greater than or equal to 150 mm and less than or equal to 190 mm, greater than or equal to 155 mm and less than or equal to 185 mm, or even greater than or equal to 160 mm and less than or equal to 180 mm.

Accordingly, embodiments of the present disclosure provide systems for a point-of-care analyzer with a sample preparation tray. The systems may include an analysis unit that receives a biological sample for analysis, a housing that at least partially surrounds the analysis unit, and a tray positioned on the housing. The analysis unit may further include an interrogation device. The interrogation device and the memory may each be communicatively coupled to the processor. The housing may additionally define a cartridge slot for receiving a cartridge comprising the biological sample for analysis. Additionally, the tray may include an upper surface for holding the cartridge and defining one or more reagent holders for holding a consumable reagent.

Other embodiments of the present disclosure provide systems for a sample preparation tray. The systems may include an upper surface and a pair of reagent holders. The upper surface of the tray may further define a cartridge holding section configured to receive and retain a cartridge containing a biological sample. Additionally, the pair of reagent holders may be provided on opposite sides of the cartridge holding section, and each reagent holder may be configured to receive and retain a consumable reagent.

It may be noted that one or more of the following claims utilize the terms “where,” “wherein,” or “in which” as transitional phrases. For the purposes of defining the present technology, it may be noted that these terms are introduced in the claims as an open-ended transitional phrase that are used to introduce a recitation of a series of characteristics of the structure and should be interpreted in like manner as the more commonly used open-ended preamble term “comprising.”

Having described the subject matter of the present disclosure in detail and by reference to specific embodiments, it may be noted that the various details described in this disclosure should not be taken to imply that these details relate to elements that are essential components of the various embodiments described in this disclosure, even in casings where a particular element may be illustrated in each of the drawings that accompany the present description. Rather, the claims appended hereto should be taken as the sole representation of the breadth of the present disclosure and the corresponding scope of the various embodiments described in this disclosure. Further, it will be apparent that modifications and variations are possible without departing from the scope of the appended claims.

What is claimed is:

1. A point-of-care analyzer comprising:
an analysis unit that receives a biological sample for analysis, the analysis unit comprising an interrogation device;
a housing at least partially surrounding the analysis unit, the housing defining a cartridge slot for receiving a cartridge comprising the biological sample; and
a tray on the housing, the tray comprising an upper surface for holding the cartridge and one or more reagent holders for holding a consumable reagent.

2. The point-of-care analyzer of claim 1, wherein the interrogation device comprises an optical microscope.

3. The point-of-care analyzer of claim 1, wherein the housing comprises a rear housing portion and a front housing portion movable relative to the rear housing portion, the front housing portion further defining the cartridge slot.

4. The point-of-care analyzer of claim 3, wherein the tray is on an upper section of the front housing portion above the cartridge slot in a vertical direction.

5. The point-of-care analyzer of claim 3, wherein the front housing portion defines a ledge formed below the cartridge slot in a vertical direction, the ledge further comprising an upper face, wherein the tray is on the upper face of the ledge.

6. The point-of-care analyzer of claim 5, wherein a distance between the tray and the cartridge slot is equal to or greater than 20 mm and less than or equal to 80 mm.

7. The point-of-care analyzer of claim 5, wherein a distance between the tray and the cartridge slot is equal to or greater than 140 mm and less than or equal to 200 mm.

8. The point-of-care analyzer of claim 1, wherein the tray is removable from the housing.

9. The point-of-care analyzer of claim 1, wherein the tray is formed as a one-piece, monolithic structure with the housing.

10. The point-of-care analyzer of claim 1, wherein the tray comprises a cartridge holding section such that the cartridge is positionable and retained within the cartridge holding section.

11. The point-of-care analyzer of claim 1, wherein the one or more reagent holders is a pair of reagent holders.

12. The point-of-care analyzer of claim 11, wherein the pair of reagent holders are positioned on opposite sides of the upper surface of the tray.

13. A housing of a point-of-care analyzer, the housing comprising a tray, the tray comprising:
an upper surface defining a cartridge holding section configured to receive and retain a cartridge comprising a biological sample; and
a pair of reagent holders provided on opposite sides of the cartridge holding section, each reagent holder configured to receive and retain a consumable reagent.

14. The tray of claim 13, wherein the tray comprises: a front edge, a rear edge opposite the 6 front edge, a first side edge extending between the front edge and the rear edge, a second side edge opposite the first side edge extending between the front edge and the rear edge, the upper surface, and a lower surface opposite the upper surface, a longitudinal axis extends between the first side edge and the second side edge.

15. The tray of claim 14, wherein the upper surface of the tray comprises a plurality of ribs, the plurality of ribs defining the cartridge holding section.

16. The tray of claim 14, wherein the upper surface of the tray comprises a recessed region defining the cartridge holding section.

17. The tray of claim 14, wherein an opening is formed in each reagent holder, wherein a consumable reagent is positionable in the opening of a respective reagent holder.

18. The tray of claim 17, wherein the openings of the pair of reagent holders are spaced apart from one another along the longitudinal axis.

19. The tray of claim 17, wherein each reagent holder includes a protrusion extending from the lower surface of the tray, the openings formed at an upper end of a respective protrusion.

20. The tray of claim 17, wherein each reagent holder includes a protrusion extending from the upper surface of the tray, the openings formed at an upper end of a respective protrusion.
